(19) United States
(12) Patent Application Publication
(10) Pub. No.: US 2003/0114924 A1
(43) Pub. Date: Jun. 19, 2003

(54) Polymer heart valve

(76) Inventor: Riyad Moe, Austin, TX (US)

Correspondence Address: Timothy L. Scott, Senior Intellectual Property Counsel, SULZER MEDICA USA INC., 3 East Greenway Plaza, Suite 1600, Houston, TX, 77046, US

(21) Appl. No.: 10/023,514

(22) Filed: Dec. 18, 2001

(51) Int. Cl. A61F 2/24

(52) U.S. Cl. 623/2.12

(57) ABSTRACT

A prosthetic heart valve comprises a valve body and a plurality of flexible leaflets. Each leaflet comprises an attachment end, attached to the valve body, and a free margin. The free margin comprises a center portion and two side portions, an inflow surface and an outflow surface. The outflow surface has a lower curvature than the inflow surface such that the center portion of the free margin has a first cross section which is greater than a second cross section of the side portions.

### BACKGROUND OF THE INVENTION

This invention relates to valves, and in particular, to flexible leaflet heart valves useful to replace valves of the heart, such as the aortic or pulmonary valve.

There are a number of medical conditions in which one or more valves of the heart are diseased, leading to insufficiency or impaired function of the affected valve. A variety of different types of prosthetic valves have been developed to replace dysfunctional mitral, aortic and pulmonary valves. These prostheses include mechanical valves, tissue valves and polymer valves.

A prosthetic valve is typically implanted into an annular opening created in the heart following removal of a diseased valve. The valve may be secured in the annulus by suturing the host tissue to a sewing ring affixed to the valve. The replacement heart valve functions essentially as a one-way check valve.

Early heart valve prostheses included ball-and-cage valves and disc-and-cage valves in which a ball or a disc was housed in a cage. One side of the cage provided an orifice through which blood flowed either into or out of the heart, depending on the valve being replaced. When blood flowed in a forward direction, the energy of the blood flow forced the ball or disc to the back of the cage allowing blood to flow through the valve. When blood attempted to flow in a reverse direction, or “regurgitate,” the energy of the blood flow forced the ball or disc into the orifice in the valve and blocked the flow of blood.

A bileaflet valve comprised an annular valve body in which two opposed leaflet occluders were pivotally mounted. The occluders were typically substantially rigid, although some designs incorporated flexible leaflets, and moved between a closed position, in which the two leaflets were mated and blocked blood flow in the reverse direction, and an open position, in which the occluders were pivoted away from each other and did not block blood flow in the forward direction. The energy of blood flow caused the occluders to move between their open and closed positions.

A trileaflet valve comprised an annular valve body in which three flexible leaflets were mounted to a portion of the valve body, called a “stent,” located at the circumference of the annulus. When blood flowed in the forward direction, the energy of the blood flow deflected the three leaflets away from the center of the annulus and allowed blood to flow through. When blood flowed in the reverse direction, the three leaflets engaged each other in a coaptive region, occluded the valve body annulus and prevented the flow of blood. The valve leaflets were made from tissue, such as specially treated porcine or bovine pericardial tissue, and more recently, from man-made materials such as polyurethane or another biocompatible polymer.

More recently, different leaflet configurations for trileaflet valves have been used in an attempt to create a physiologic valve. For example, U.S. Pat. No. 5,500,016 to Fisher and WO 98/32400 each describe artificial heart valves, in which the free margin of each leaflet has a smooth sweeping (e.g., curved) trajectory on the inflow and outflow surfaces, and a uniform cross section.

Leaflets having such a design cannot close without considerable bending stresses at the triple point, where the three leaflets come together in the closed position. In order for the valve to close, each of the leaflets must bend to a sharp point, thereby crushing the leaflet material on the outflow, and stretching the material on the inflow. Alternatively, if the leaflet cannot bend to a tight radius, the triple point remains open, and the valve does not seal. As a result, this design provides a sub-optimal seal. Further, high bending stresses reduce the life of the valve.

Another trileaflet valve design incorporates parallel leaflet ends. U.S. Pat. No. 4,7789,461 to Pietch discloses a heart valve having leaflet ends that are both vertical and parallel. There is a bead along the free margin of each leaflet, and the cross section is constant throughout the entire length of the free margin. There is a kink in the free margin at the triple point. The leaflets are formed with the inflow surfaces parallel to each other and in close proximity. In this design, the leaflets do not need to change their shape for the valve to close. Thus, there is minimal or no bending stress when the valve is closed. However, the leaflets may undergo substantial stress when the valve is opened.

There is therefore a need for a heart valve that seals and opens effectively without creating undue stresses on the leaflet components in the open or closed positions.

### SUMMARY OF THE INVENTION

The present invention overcomes the problems and disadvantages associated with current strategies and designs and provides a prosthetic heart valve that seals effectively, and is designed so that there are no undue stresses on the valve in the open or closed position. The invention provides a valve having superior sealing and increased durability and longevity over previous designs.

Accordingly, one embodiment of the invention is directed to a heart valve comprising a valve body and a plurality of leaflets. Each leaflet comprises a free margin, having an inflow surface and an outflow surface, and an attachment end attached to the valve body. The free margin of each leaflet has two side portions and a center portion disposed between the two side portions. The two side portions of each free margin are substantially non-co-planar and substantially non-parallel. The outflow surface of each side portion of each free margin is substantially parallel to the corresponding inflow surface of that side portion. The adjacent inflow surfaces of the side portions of adjacent leaflets are substantially parallel. The inflow surface of the center portion of each free margin is a transition between the inflow surfaces of the respective two side sections of said free margin. The outflow surface of the center portion of each free margin is a transition between the outflow surfaces of the respective two side sections of said free margin. The inflow surface of the center portion has a higher curvature than the outflow surface of the corresponding center portion of said free margin.

Another embodiment of the invention is directed to a prosthetic heart valve comprising a valve body and a plurality of flexible leaflets. Each leaflet comprises an attachment end, attached to the valve body, and a free margin. The free margin comprises a center portion and two side portions, an inflow surface and an outflow surface. The inflow surface comprises a first flat face, a second flat face, and a leaflet center. The first and second flat faces intersect at the leaflet center. The outflow surface comprises a curved surface disposed with respect to the inflow surface such that the center portion of the free margin has a first cross section which is greater than a second cross section of the side portions. The heart valve further has an open and closed position such that when the valve is in the closed position, the first flat face of one of the plurality of leaflets is disposed adjacent and parallel to the second flat face of another one of the plurality of leaflets and the second flat face of the one of the plurality of leaflets is disposed adjacent and parallel to the first flat face of the another one of the plurality of leaflets.

Another embodiment of the invention is directed to a method for forming a flexible heart valve comprising the steps of: molding a one piece valve comprising a valve body and a plurality of flexible movable leaflets; and forming each leaflet to include an attachment end, attached to the valve body, and a free margin. The free margin comprises a center portion and two side portions, an inflow surface and an outflow surface. The inflow surface comprises a first flat face, a second flat face, and a leaflet center, the first and second flat faces intersecting at the leaflet center. The outflow surface comprises a curved surface disposed with respect to the inflow surface such that the center portion of the free margin has a first cross section which is greater than a second cross section of the side portions. The heart valve further has an open and closed position wherein when the valve is in the closed position, the first flat face of one of the plurality of leaflets is disposed adjacent and parallel to the second flat face of another one of the plurality of leaflets and the second flat face of the one of the plurality of leaflets is disposed adjacent and parallel to the first flat face of the another one of the plurality of leaflets.

Still another embodiment is directed to a method for occluding and permitting fluid flow between a first heart chamber and a second heart chamber or vessel comprising providing a prosthetic heart valve according to the invention between the first heart chamber and the second heart chamber or vessel.

Other embodiments and advantages of the invention are set forth in part in the description which follows, and in part, will be obvious from this description, or may be learned from the practice of the invention.

### DESCRIPTION OF THE DRAWINGS

FIG. 1 is a perspective outflow view of a valve according to one embodiment of the invention.

FIG. 2 is a plan view of the valve shown in FIG. 1.

FIG. 3 is a cross sectional view of the valve of FIG. 1 taken along line “A-A” of FIG. 2.

FIG. 4 is a cross sectional view of the valve of FIG. 1 taken along line “B-B” of FIG. 2.

FIG. 5 is an enlarged detail view of the center of the valve designated “C” in FIG. 1.

FIG. 6 is a plan view of another embodiment of the invention showing an alternate configuration of the free margin.

### DETAILED DESCRIPTION OF THE INVENTION

As embodied and broadly described herein, the present invention relates to flexible leaflet heart valves. Specifically, the present invention is directed to a trileaflet valve in which the inflow surface of the free margin or top edge of each leaflet is parallel and in close proximity to the inflow surface of the free margin or top edge of the adjacent leaflet in the region of the leaflet center, or triple point, when the valve is in the closed position. The outflow surface of the free margin sweeps broadly from one side of the leaflet to the other. The inflow surface of the center portion of the free margin has a higher curvature than the outflow surface, so that the center of the free margin is thicker than the sides.

The parallel inflow surfaces give the benefit of zero, or near zero, bending stress in the closed position. In addition, the thicker free margin at the leaflet center reduces stresses of the open valve in two ways. First, the peak stress needed to resist the bending moment arising from the pressure load is reduced. Second, the neutral surface is not kinked in either the open or closed position. Because bending stress is proportional to change in local curvature, and curvature is the reciprocal of radius, the absolute stresses are reduced by keeping the neutral surface away from a zero radius. The thicker leaflet of the invention has lower stress under a constant bending moment.

The present invention avoids the problems in existing valves having a free margin with both a curved inflow and outflow surface and uniform cross section. Leaflets having such a curved free margin cannot close without considerable bending stresses at the triple point, i.e., the point where the three leaflets come together in the closed position. In order for the valve to close, each of the leaflets must bend to a sharp point, i.e., zero radius. Bending stress in a membrane is related to the local change in curvature of the membrane under deformation. Curvature is the reciprocal of radius. So, as the radius approaches zero at the center of the leaflet, the curvature approaches infinity. As such, bending stresses are extremely high regardless of the original curvature or the thickness of the membrane. Further, the high bending stresses reduce the life of the valve.

Closure of valves having a curved free margin on the inflow surface is not physically possible without crushing the leaflet material. Alternatively, if the leaflet cannot bend to a tight radius, the triple point remains open, and the valve does not seal. As a result, this design provides a sub-optimal seal.

In this manner, the bending stresses are, in theory, independent of leaflet thickness. Simply thickening the leaflet or providing a bead at the free margin does not rectify the problems inherent in the foregoing design. If the leaflet were thickened, the closure problem would only be exacerbated. A thicker leaflet with more bending resistance would leak even more than a thin leaflet. Further, if the thicker leaflet is forced to bend down to the tight radius of the thin leaflet in an attempt to close the valve, the bending stresses are raised. Bending stresses are a function of the distance of the outermost material particles from the neutral surface of the leaflet. The neutral surface is the imaginary surface that undergoes no strain in bending. It is usually close to the center line of a leaflet if the material is homogenous.

The present invention also avoids problems in existing valves having free margins with parallel and vertical inflow and outflow surfaces. Although such valves are able to close without having to change shape, they undergo excess stresses in the open position. There is a kink in the free margin at the triple point. The leaflets are formed with the inflow surfaces parallel to each other and in close proximity. In this design, the leaflets do not need to change their shape for the valve to close. Thus, there are minimal or no bending strains or bending stress when the valve is closed. However, the inflow surface and the outflow surface, and thus the neutral surface, are all kinked at the triple point. As a result, the leaflets undergo substantial stress when the valve is opened. Either the triple point of the leaflet is completely unfurled, so that there is a smooth sweep across the triple point, or the kinked nature of the triple point is retained in the open leaflet.

In the first case, when the triple point is unfurled, theoretically infinite stress is present. In a membrane with no curvature in the unstressed state, the bending strain under load is proportional to the resulting curvature of the membrane. This is shown by the following formula:

1/ρ=K=−ε/y

where ρ is the radius of curvature;

K is curvature;

ε is the linear strain in the strained fibers at a distance y from the neutral surface; and

y is the distance from the neutral surface to the strained fibers.

(See E. P. Popov, Mechanics of Materials, Second Edition, Prentice-Hall, Inc., 1976, page 355, Equation 11-5.)

In the more general case, in a membrane of given curvature in the unstressed state, the bending strain under load is proportional to the change in curvature of the membrane. Therefore, if a leaflet has a kink, or a near-zero radius, in its unstressed state, then it has near infinite curvature in its unstressed state. If the kink is unfurled, then the local radius is significantly increased and, thereby the local curvature reduced to finite values. The change of curvature from near infinite to finite is itself nearly infinite. Because the change in curvature is near infinite, strain is nearly infinite, and so is stress.

In the second case, the kink is not worked out and high bending stresses may not necessarily be present. However, the pressure load that holds the valve open imparts a bending moment at the triple point. The peak stress required to resist a bending moment in a beam is a function of the modulus of the material and the distance of the outermost fibers from the neutral surface. This is shown by the following formula:

σ=−My/I

where σ is the normal stress at a section;

M is the internal or resisting bending moment;

y is the distance from the neutral axis of the beam to the point on the section where the normal stress σ is wanted measured perpendicular to the neutral axis; and

I is the moment of inertia of the whole cross-sectional area of the section about its neutral axis.

(See E. P. Popov, Mechanics of Materials, Second Edition, Prentice-Hall, Inc., 1976, page 126, Equation 5-1 a.)

As a result, thin beams see higher peak stress to resist the same moments. Thick beams see lower peak stress for the same moment resistance. In this case, stress in the open position is related to thickness. Stress in the closed position is related to shape. Providing a thicker free margin will reduce the magnitude of the peak bending stress. However, the thicker free margin is only needed in regions undergoing significant bending, i.e., near the center of the free margin. Any increase in thickness reduces the flexibility of the leaflet and increases its resistance to forward fiow. It would produce unecessary leaflet stiffness to thicken the entire length of the free margin.

Unlike the foregoing designs, valves according to the present invention seal effectively and simultaneously avoid excess stress on the leaflets, whether the valve is in the fully open or fully closed positions.

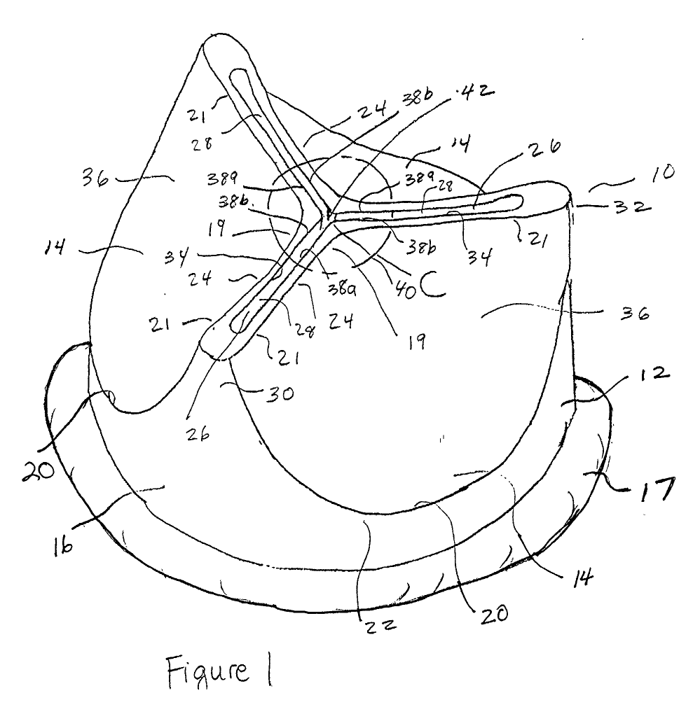

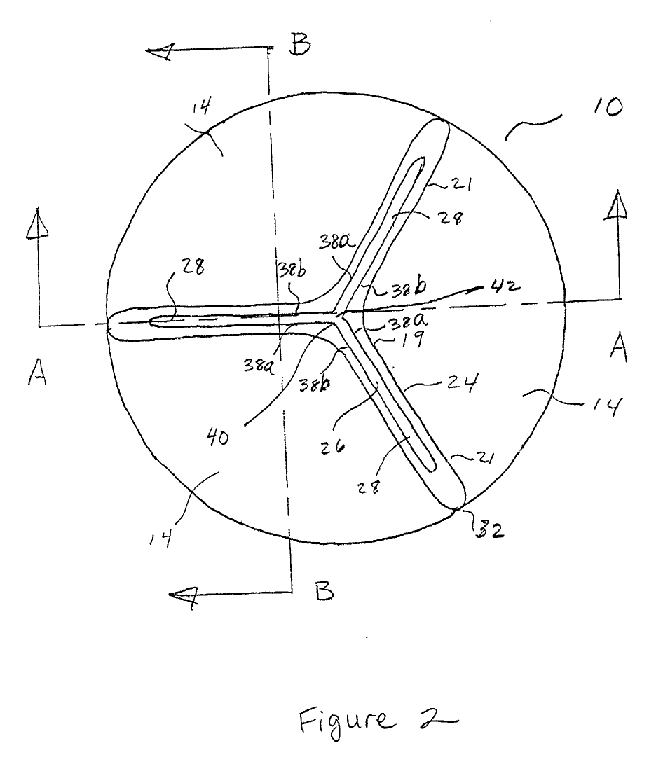

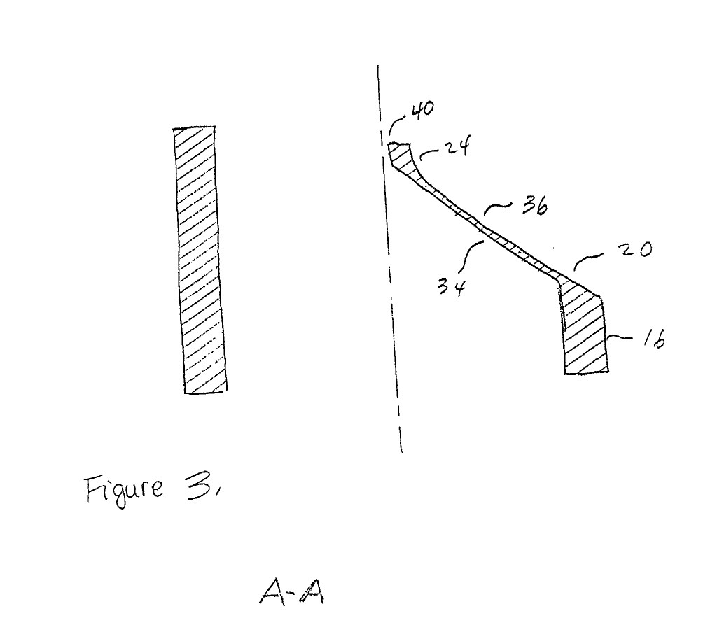

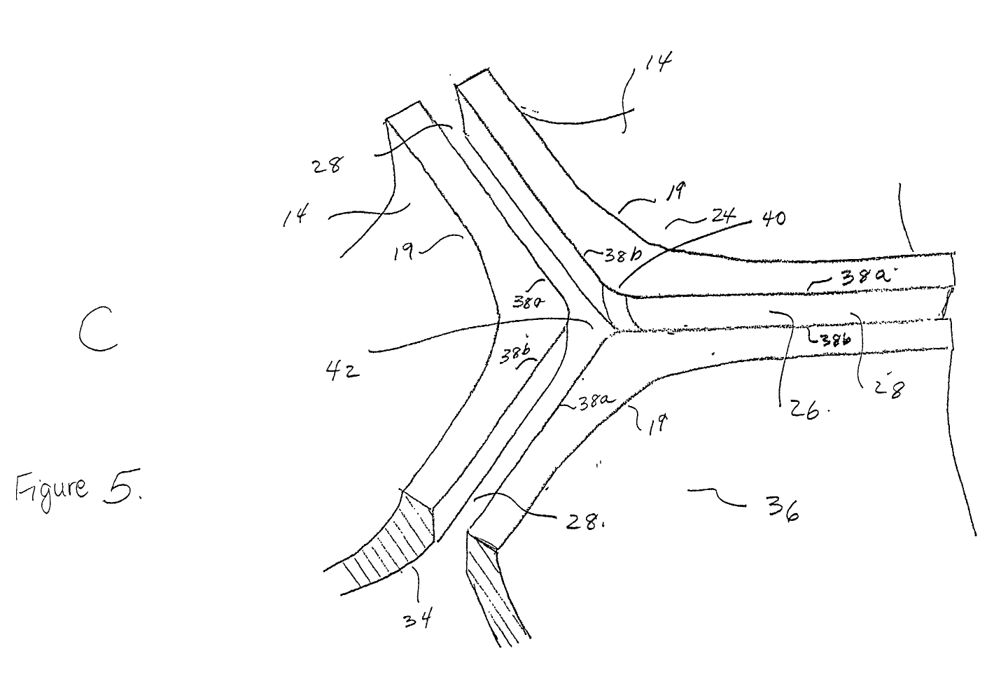

A preferred heart valve according to the invention is schematically depicted in FIGS. 1-5. Referring to FIGS. 1-5, trileaflet valve 10 comprises an annular valve body 12, and three flexible leaflets 14. Although any suitable material and manufacturing process may be used, body 12 and leaflets 14 are preferably formed as a one piece molded biocompatible polymer, such as silicone or polyurethane. Annular valve body 12 preferably has a base 16. A sewing ring 17 may be formed with base 16. Each individual leaflet 14 has a curved attachment end 20 which extends from attachment curve 22 of body 12. Each leaflet also has a top edge or free margin 24 that is not attached to the valve body. Free margin 24 has a center portion 19 and two side portions 21. The center portion 19 is a transition between and is disposed between the two side portions 21. The two side portions 21 are substantially non-coplanar and substantially non-parallel.

Preferably, the heart valve is molded to include a gap 26, comprising three legs 28, formed between the free margins 24 of the three leaflets.

Attachment curves 22 define the coupling between curved attachment ends 20 of the leaflets and the body, 12, and also define three shaped posts, 30, which are extensions of body 12. Each post 30 terminates at commissure 32 where one leg 28 of gap 26 ends.

Free margin 24 of each leaflet has an inflow surface 34 and an outflow surface 36. The outflow surface and inflow surface of the side portions of each free margin are generally parallel to each other. The inflow surface of the center portion has a higher curvature than the outflow surface of the center portion. As a result the center portion is thicker than the side portions. Preferably, the radius of curvature of the inflow surface is between about 0.005 inches to about 0.020 inches and the radius of curvature of the outflow surface is about 0.050 to about 0.250 inches. More preferably, the radius of curvature of the inflow surface is approximately 0.020 inches and the radius of curvature of the outflow surface is 0.100 inches.

For example, in the preferred embodiment depicted in FIGS. 1-5, the inflow surface of center portion 19 of free margin 24 comprises two flat faces 38a and 38b disposed at an angle to each other. In the trileaflet valve shown in FIGS. 1-5, the faces would lie at an angle of approximately 120 degrees to each other. Flat faces 38a and 38b intersect at leaflet center 40. As shown in FIG. 1, when valve 10 is in the closed position or in the neutral position, face 38a of each leaflet 14 lies adjacent and generally parallel to face 38b of the adjacent leaflet, and face 38b lies adjacent and generally parallel to face 38a of the adjacent leaflet on the other side. When the valve is in the neutral position, the faces 38a and 38b of each leaflet are separated from each other by gap 26. When the valve is in the fully closed position, gap 26 is essentially eliminated. Triple point 42 is formed at the central point where the three leaflet centers 40 come together.

The outflow surface of center portion 19 of free margin 24 is curved in shape. It has a smaller curvature than the inflow surface. As a result, the cross section of center portion 19 of free margin 24 is thicker than the cross sections of side portions 21. The cross section of the free margin is thickest at triple point 42. The curved surface of the outflow surface may assume a variety of forms such as a cone, conic cylinder, ellipsoid or paraboloid. Conic cylinders include, but are not limited to, circular cylinders, hyperbolic cylinders, parabolic cylinders, and elliptic cylinders. Preferably, the center portions are fillets of constant radii. Preferably, each leaflet is identical or substantially identical.

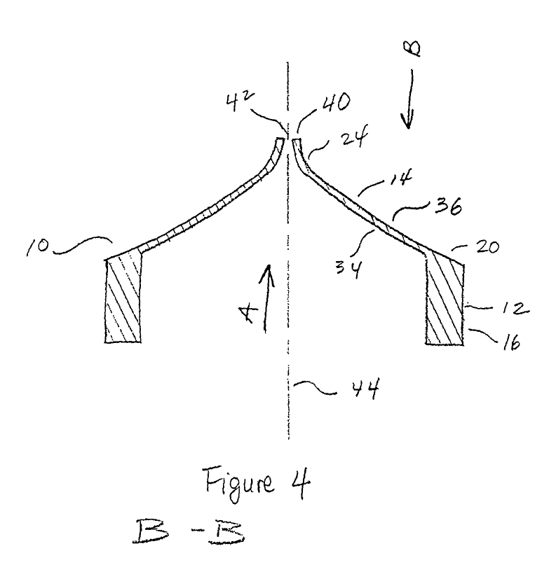

When fluid flow is in the forward, or inflow direction, i.e., in the direction of arrow A shown in FIG. 4, the pressure of the blood flow causes the leaflets 14 to deflect away from a central longitudinal axis 44 of the valve body into an open position. In this open position, the leaflets define a large flow orifice, which presents little resistance to fluid flow.

When fluid attempts to flow in the reverse direction, i.e., in the direction of arrow B shown in FIG. 4, the flow causes the leaflets to deflect towards axis 44. In this closed position, gap 26 is essentially eliminated, and faces 38a and 38b of leaflet 14 engage the corresponding faces of the adjoining leaflets, and form coaptive areas that help the valve seal against reverse flow.

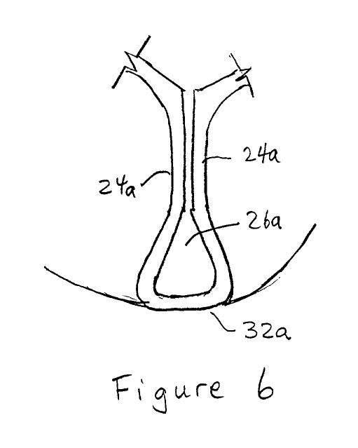

As shown in FIG. 6, in an alternate embodiment of the invention, free margin 24a of each leaflet may be modified so that the the free margin is angled away from the free margin of its opposing leaflet as it approaches the commisure, such that gap 26a is widest near commissure 32a.

Accordingly, one embodiment of the invention is directed to a heart valve comprising a body and a plurality of leaflets. Each leaflet comprises a free margin, having an inflow surface and an outflow surface, and an attachment end attached to the body. The free margin of each leaflet has two side portions and a center portion disposed between the two side portions. The two side portions of each free margin are substantially non-co-planar and substantially non-parallel. The outflow surface of each side portion of each free margin is substantially parallel to the corresponding inflow surface of that side portion. The adjacent inflow surfaces of the side portions of adjacent leaflets are substantially parallel. The inflow surface of the center portion of each free margin is a transition between the inflow surfaces of the respective two side sections of said free margin. The outflow surface of the center portion of each free margin is a transition between the outflow surfaces of the respective two side sections of said free margin. The inflow surface of the center portion has a higher curvature than the outflow surface of the corresponding center portion of said free margin. Although valves according to the invention may have three or more leaflets, in a preferred embodiment, the valve has three leaflets.

Another embodiment is directed to a prosthetic heart valve comprising a valve body and a plurality of flexible leaflets. Each leaflet comprises an attachment end, attached to the valve body, and a free margin. The free margin comprises a center portion and two side portions, an inflow surface and an outflow surface. The inflow surface comprises a first flat face, a second flat face, and a leaflet center. The first and second flat faces intersect at the leaflet center. The outflow surface comprises a curved surface disposed with respect to the inflow surface such that the center portion of the free margin has a first cross section which is greater than a second cross section of the side portions. The heart valve further has an open and closed position such that when the valve is in the closed position, the first flat face of one of the plurality of leaflets is disposed adjacent and parallel to the second flat face of another one of the plurality of leaflets and the second flat face of the one of the plurality of leaflets is disposed adjacent and parallel to the first flat face of the another one of the plurality of leaflets. Preferably, the first cross section has a thickness of between about 0.020 inches to about 0.040 inches and the second cross section has a thickness of between about 0.005 inches and about 0.015 inches.

In a further embodiment, a method of forming a flexible heart valve includes molding a one piece valve comprising a valve body and a plurality of flexible movable leaflets and forming each leaflet to include an attachment end, attached to the valve body, and a free margin. The free margin comprises a center portion and two side portions, an inflow surface and an outflow surface. The inflow surface comprises a first flat face, a second flat face, and a leaflet center. The first and second flat faces intersect at the leaflet center. The outflow surface comprises a curved surface disposed with respect to the inflow surface such that the center portion of the free margin has a first cross section which is greater than a second cross section of the side portions. The heart valve further has an open and closed position wherein when the valve is in the closed position, the first flat face of one of the plurality of leaflets is disposed adjacent and parallel to the second flat face of another one of the plurality of leaflets and the second flat face of the one of the plurality of leaflets is disposed adjacent and parallel to the first flat face of the another one of the plurality of leaflets. Still another embodiment is directed to a method for occluding and permitting fluid flow between a first heart chamber and a second heart chamber or vessel comprising providing a prosthetic heart valve according to the invention between the first heart chamber and the second heart chamber or vessel.

Other embodiments and uses of the invention will be apparent to those skilled in the art from consideration of the specification and practice of the invention disclosed herein. It is intended that the specification and examples be considered exemplary only, with the true scope and spirit of the invention indicated by the following claims. Variations and modifications of the elements of the claimed invention will be apparent to persons skilled in the art from a consideration of this specification or practice of the invention disclosed herein.

What is claimed is:

1. A heart valve comprising:
a valve body; and
a plurality of leaflets, each leaflet comprising a free margin, the free margin comprising an inflow surface and an outflow surface, and an attachment end attached to the valve body, the free margin of each leaflet further comprising
two side portions and a center portion disposed between the two side portions, the two side portions each being substantially non-co-planar and substantially non-parallel,
the outflow surface of each side portion of said free margin being substantially parallel to the corresponding inflow surface of said side portion,
the adjacent inflow surfaces of the side portions of adjacent leaflets being substantially parallel,
the inflow surface of the center portion of each free margin being a transition between the inflow surfaces of the respective two side sections of said free margin,
the outflow surface of the center portion of each free margin being a transition between the outflow surfaces of the respective two side sections of said free margin, and
the inflow surface of the center portion having a higher curvature than the outflow surface of the corresponding center portion of said free margin.

2. The valve of claim 1 wherein the center portions are fillets of constant radii.

3. The valve of claim 1 wherein said valve has three leaflets.

4. The valve of claim 1 wherein each leaflet is substantially identical.

5. A prosthetic heart valve comprising:
a valve body; and a plurality of flexible leaflets, each leaflet comprising an attachment end attached to said valve body and a free margin, wherein said free margin comprises a center portion and two side portions, an inflow surface and an outflow surface, said inflow surface comprising a first flat face, a second flat face, and a leaflet center, said first and second flat faces intersecting at said leaflet center, and wherein said outflow surface comprises a curved surface disposed with respect to said inflow surface such that the center portion of the free margin has a first cross section which is greater than a second cross section of the side portions, said heart valve further having an open and closed position wherein when the valve is in the closed position, the first flat face of one of said plurality of leaflets is disposed adjacent and parallel to the second flat face of another one of said plurality of leaflets and the second flat face of said one of said plurality of leaflets is disposed adjacent and parallel to the first flat face of the another one of said plurality of leaflets.

6. The valve of claim 5 wherein the valve has three leaflets.

7. The valve of claim 5 wherein the first cross section has a thickness of between about 0.020 inches to about 0.040 inches and the second cross section has a thickness of between about 0.005 inches and about 0.015 inches.

8. The valve of claim 5 wherein the outflow surface comprises a conic cylinder.

9. The valve of claim 8 wherein the conic cylinder comprises a circular cylinder, a hyperbolic cylinder, a parabolic cylinder or an elliptic cylinder.

10. The valve of claim 5 wherein each leaflet is substantially identical.

11. A method for forming a flexible heart valve comprising the steps of:
molding a one piece valve comprising a valve body and a plurality of flexible movable leaflets; and
forming each leaflet to include an attachment end attached to said valve body and a free margin, wherein said free margin comprises a center portion and two side portions, an inflow surface and an outflow surface, said inflow surface comprising a first flat face, a second flat face, and a leaflet center, said first and second flat faces intersecting at said leaflet center, and wherein said outflow surface comprises a curved surface disposed with respect to said inflow surface such that the center portion of the free margin has a first cross section which is greater than a second cross section of the side portions, said heart valve further having an open and closed position wherein when the valve is in the closed position, the first flat face of one of said plurality of leaflets is disposed adjacent and parallel to the second flat face of another one of said plurality of leaflets and the second flat face of said one of said plurality of leaflets is disposed adjacent and parallel to the first flat face of the another one of said plurality of leaflets.

12. The method of claim 11 wherein said valve comprises three leaflets.

13. The method of claim 11 wherein the first cross section has a thickness of between about 0.020 inches to about 0.040 inches and the second cross section has a thickness of between about 0.005 inches and about 0.015 inches.

14. The method of claim 11 wherein the outflow surface comprises a conic cylinder.

15. The method of claim 14 wherein the conic cylinder comprises a circular cylinder, a hyperbolic cylinder, a parabolic cylinder or an elliptic cylinder.

16. The method of claim 11 wherein each leaflet is substantially identical.

17. A method for occluding and permitting fluid flow between a first heart chamber and a second heart chamber or vessel comprising:
providing a prosthetic heart valve between said first heart chamber and said second heart chamber or vessel, said prosthetic heart valve comprising a valve body and a plurality of flexible leaflets, each leaflet comprising an attachment end attached to said valve body and a free margin, wherein said free margin comprises a center portion and two side portions, an inflow surface and an outflow surface, said inflow surface comprising a first flat face, a second flat face, and a leaflet center, said first and second flat faces intersecting at said leaflet center, and wherein said outflow surface comprises a curved surface disposed with respect to said inflow surface such that the center portion of the free margin has a first cross section which is greater than a second cross section of the side portions, said heart valve further having an open and closed position wherein when the valve is in the closed position, the first flat face of one of said plurality of leaflets is disposed adjacent and parallel to the second flat face of another one of said plurality of leaflets and the second flat face of said one of said plurality of leaflets is disposed adjacent and parallel to the first flat face of the another one of said plurality of leaflets.

18. The method of claim 17 wherein said valve comprises three leaflets.

19. The method of claim 17 wherein the outflow surface comprises a conic cylinder.

20. The method of claim 19 wherein the conic cylinder comprises a circular cylinder, a hyperbolic cylinder, a parabolic cylinder or an elliptic cylinder.

21. The method of claim 17 wherein each leaflet is substantially identical.
